(19) United States
(12) Patent Application Publication
(10) Pub. No.: US 2008/0249477 A1
(43) Pub. Date: Oct. 9, 2008

(54) PEN INJECTOR HAVING A NEEDLE SHIELD

(75) Inventors: John Paproski, North Wales, PA (US); Paul Norton, Trumbauersville, PA (US)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: WEST PHARMACEUTICAL SERVICES, INC., Lionville, PA (US)

(21) Appl. No.: 12/062,264

(22) Filed: Apr. 3, 2008

Related U.S. Application Data
(60) Provisional application No. 60/910,331, filed on Apr. 5, 2007.

(51) Int. Cl. A61M 5/32

(52) U.S. Cl. 604/198

(57) ABSTRACT

A pen injector includes a vial cartridge and a generally tubular body that surrounds the vial cartridge. A needle shield assembly is removably mounted to a mounting end of the body and includes a hollow needle that has a tip for injecting into a patient. A needle hub at least partially surrounds the hollow needle and a slideable sleeve is slideably mounted on the needle hub. The needle hub has an outer surface which deflects the slideable sleeve as the slideable sleeve slides toward a receiving end of the needle hub and into a retracted position. The slideable sleeve bears resiliently on the outer surface of the needle hub during sliding movement of the slideable sleeve relative to the needle hub toward the retracted position such that a restoring force is generated for urging the slideable sleeve to move toward an injection end of the needle hub and into an extended position in which the tip of the needle is located completely inside of the slideable sleeve.

### CROSS-REFERENCE TO RELATED APPLICATIONS

This application claims the benefit of U.S. Provisional Patent Application No. 60/910,331 filed Apr. 5, 2007 entitled “Pen Applicator with Needle Shield” which is incorporated by reference herein in its entirety.

### BACKGROUND OF THE INVENTION

The present invention relates to the incorporation of a passive needle protection system for needles used with pen type medical injection systems (pen injectors).

Diabetics and others frequently find themselves in situations where the assistance of a health professional to administer subcutaneous or intra muscular injections of measured amounts of a liquid agent is generally not available. In such situations such persons need to have a low cost syringe which does not require the assistance of a healthcare professional for an injection to achieve the desired measure of accuracy. It is often the case that such persons require more than one dose per day, each dose being of different volumes. Dispensers of this general type are known and have the general appearance of a pen or mechanical pencil. Such dispensers, known as pen applicators or injectors, are typically large enough to hold several such doses, yet are small enough to fit conveniently in one's pocket or purse. Though a removable cap is usually included, accidental needle sticks can occur when the needle is not in use or when replacing cap on the needle. Additionally, conventional pen injectors permit the re-use of needles.

Various safety or needle shield systems have been developed and proposed for conventional hypodermic syringes. Such needle shield systems may include a tubular shield that is spring biased to enclose the needle cannula and lock in the extended enclosed position following injection. Such needle shield systems for conventional hypodermic syringes are operated manually, requiring an additional action, such as a twist or other force, to activate the needle shield.

It would therefore be desirable to provide pen injector with a needle shield that automatically exposes the needle when the needle is inserted in the patient. The act of removing a needle from the patient would allow the needle shield to automatically extend and lock the shield over the exposed needle.

### BRIEF SUMMARY OF THE INVENTION

Briefly stated, the present invention is directed to a pen injector that includes a vial cartridge and a generally tubular body that surrounds the vial cartridge. The body has a mounting end and a driving end. A needle shield assembly is removably mounted to the mounting end of the body. The needle shield assembly includes a hollow needle that has a tip for injecting into a patient. A needle hub at least partially surrounds the hollow needle. A slideable sleeve is slideably mounted on the needle hub. The needle hub and the slideable sleeve each have a receiving end which is distal to the tip of the needle and an injection end which is proximal to the tip of the needle. The receiving end of the needle hub is connected to the mounting end of the body and the slideable sleeve is slideable relative to the hub in the direction of the length of the needle between an extended position in which the tip of the needle is located completely inside the slideable sleeve and a retracted position in which the tip of the needle projects outwardly from the slideable sleeve. The needle hub has an outer surface which deflects the slideable sleeve as the slideable sleeve slides toward the receiving end of the needle hub and into the retracted position. The slideable sleeve bears resiliently on the outer surface of the needle hub during sliding movement of the slideable sleeve relative to the needle hub toward the retracted position such that a restoring force is generated for urging the slideable sleeve to move toward the injection end of the needle hub and into the extended position.

In another aspect, the invention is directed to a pen injector that includes a vial cartridge that has a piston. A generally tubular body surrounds the vial cartridge and has a mounting end and a driving end. The driving end includes a knob for setting a dosage level and driving the piston in accordance with the set dosage level. A needle shield assembly is removably mounted to the mounting end of the body. The needle shield assembly includes a hollow needle that has a first tip for injecting into a patient and a second tip for that pierces the vial cartridge. A needle hub at least partially surrounds the hollow needle. A slideable sleeve includes a plurality of cantilever arms that are slideably mounted on the needle hub. The needle hub and the slideable sleeve each have a receiving end which is distal to the first tip of the needle and an injection end which is proximal to the second tip of the needle. The receiving end of the needle hub is connected to the mounting end of the body. The cantilever arms are slideable relative to the hub in the direction of the length of the needle between an extended position in which the first tip of the needle is located completely inside the slideable sleeve and a retracted position in which the first tip of the needle projects outwardly from the slideable sleeve. The needle hub has a tapered outer surface which deflects the cantilever arms as the slideable sleeve slides toward the receiving end of the needle hub and into the retracted position. The cantilever arms bear resiliently on the outer surface of the needle hub during sliding movement of the slideable sleeve relative to the needle hub toward the retracted position such that a restoring force is generated for urging the slideable sleeve to move toward the injection end of the needle hub and into the extended position.

### BRIEF DESCRIPTION OF THE SEVERAL VIEWS OF THE DRAWINGS

The foregoing summary, as well as the following detailed description of a preferred embodiment of the invention, will be better understood when read in conjunction with the appended drawings. For the purpose of illustrating the invention, there is shown in the drawings an embodiment which is presently preferred. It should be understood, however, that the invention is not limited to the precise arrangements and instrumentalities shown.

In the drawings:

FIG. 1 is a side elevational view of an assembled pen injector having a covered needle shield assembly in accordance with a preferred embodiment of the present invention;

FIG. 2 is an exploded side elevational view of the pen injector including a needle shield assembly and cover shown in FIG. 1;

FIG. 3 is a partial side cross-sectional view of the needle shield assembly and cover of the pen injector shown in FIG. 1;

FIG. 4A is a side elevational view of the needle shield assembly and cover shown in FIG. 3 in the storage position or initial extended position;

FIG. 4B is a cross sectional view of the needle shield assembly and cover taken along line B-B in FIG. 4A;

FIG. 4C is a side elevational view of the needle shield assembly and cover shown in FIG. 3 in a set or intermediate position;

FIG. 4D is a cross sectional view of the needle shield assembly and cover taken along line D-D in FIG. 4C;

FIG. 4E is a side elevational view of the needle shield assembly shown in FIG. 3 with the cover removed and in the fully retracted position and a needle extending in a patient's skin;

FIG. 4F is a cross sectional view of the needle shield assembly taken along line F-F in FIG. 4E;

FIG. 4G is a side elevational view of the needle shield assembly shown in FIG. 3 with the cover removed and in the final extended position; and

FIG. 4H is a cross sectional view of the needle shield assembly taken along line H-H in FIG. 4G.

### DETAILED DESCRIPTION OF THE INVENTION

Certain terminology is used in the following description for convenience only and is not limiting. The words “right”, “left”, “lower” and “upper” designate directions in the drawings to which reference is made. The words “inwardly” and “outwardly” refer to directions toward and away from, respectively, the geometric center of a pen injector having a needle shield in accordance with the present invention, and designated parts thereof. Unless specifically set forth herein, the terms “a”, “an” and “the” are not limited to one element but instead should be read as meaning “at least one”. The terminology includes the words noted above, derivatives thereof and words of similar import.

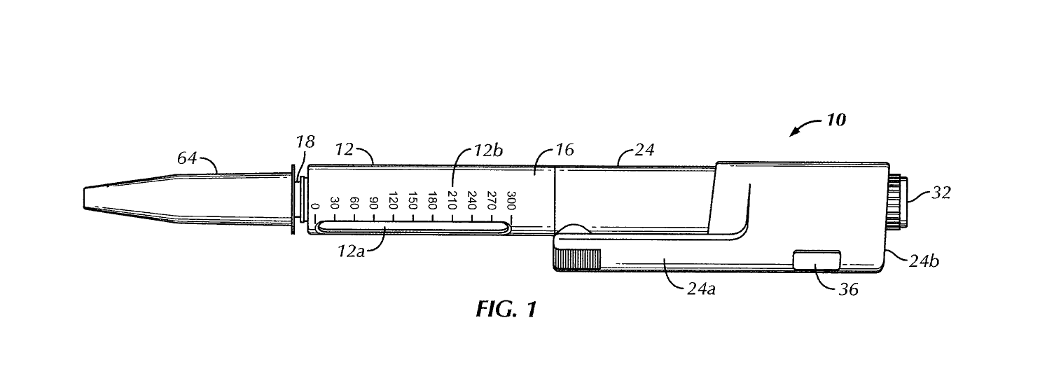

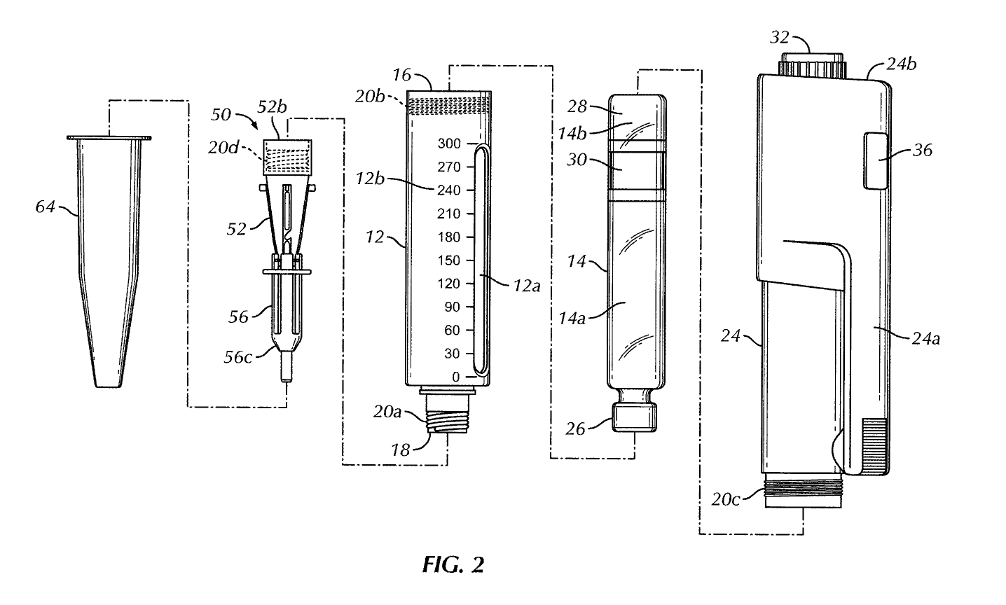

Referring to the drawings in detail, wherein like reference numerals indicate like elements throughout, there is shown in FIGS. 1-4H a preferred embodiment of a pen applicator or injector, generally designated 10, having a needle shield assembly 50 in accordance with the present invention. As shown in FIGS. 1 and 2, the needle shield assembly 50 of this invention is particularly but not exclusively adapted for pen injectors 10 similar to the Opti-Pen® which is manufactured by Disetronic AG, of Burgdorf, Switzerland, and which is commercially available through Hoechst of Frankfurt, Germany. As will be understood, however, the needle shield assembly 50 of this invention may also be used with other pen injectors similar to the pen injector 10 and with conventional hypodermic syringes and any other deliver devices. The pen injector 10 is not limited to the specific pen injector 10 shown and described.

Referring to FIGS. 1 and 2, the pen injector 10 preferably includes a body or vial holder 12 into which a vial cartridge 14 is removably or replaceably received. The vial holder 12 is preferably an elongated, generally hollow, tubular structure with a proximal end 16 and a distal end or mounting end 18. However, the vial holder 12 may have any shape and may be unreleasably attached to the vial cartridge 14. The vial holder 12 preferably has a transparent viewing window 12a and volume or dose markings 12b preferably on an outer surface proximate to the view window 12a. The vial cartridge 14 is preferably a disposable insert that contains a volume of liquid 14a or any other medication, such as insulin, for inserting into a patient. The vial cartridge 14 includes a distal end 26 that has a pierceable elastomeric seal (not shown). A proximal end 28 of the vial cartridge 14 includes a piston 30 slideably disposed in fluid tight engagement with the interior surface of a cylindrical wall 14b of the vial cartridge 14.

The pen injector 10 includes a dose setting apparatus 24. The dose setting apparatus 24 includes an adjustable knob 32 at a driving end 24b of the pen injector 10 for setting a dose of the liquid 14a to be delivered to the patient. A display 36 is preferably provided proximate to the knob 32 for indicating the dosage level set by the knob 32. Turning the knob 32 sets the dosage to a selected amount which may be displayed on the side of the dose setting apparatus 24 through or by the display 36. The display 36 is preferably a window exposing a mechanical setting such as a printed number on a rotatable wheel (not shown) operably connected to the knob 32, but the display 36 may be any suitable device for displaying the dose setting such as a digital screen. Depressing the knob 32 urges the piston 30 to move toward the pierceable elastomeric seal and dispenses the selected dose of liquid 14a through the distal end 26 of the vial cartridge 14 in accordance with the dosage setting. The dose setting apparatus 24 works in a manner similar to a retractable writing pen and the various methods for urging movement of the piston 30 are known in the art. The details of the inner workings of the dose setting apparatus 24 are omitted for convenience only and the omission of further details is not limiting. The dose setting apparatus 24 also preferably includes a clip 24a for securing the pen injector 10 to an article of clothing (not shown).

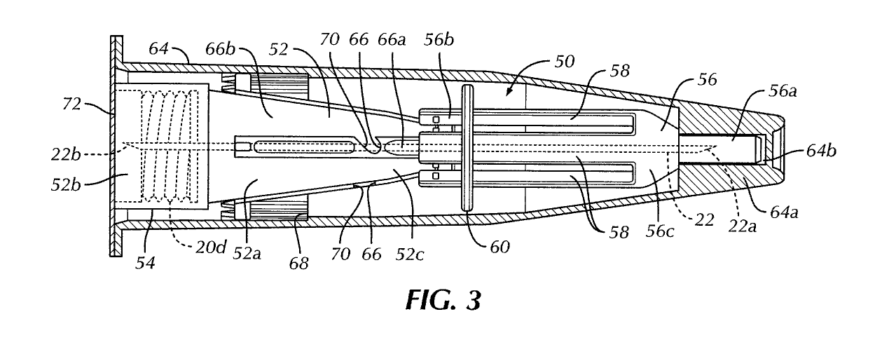

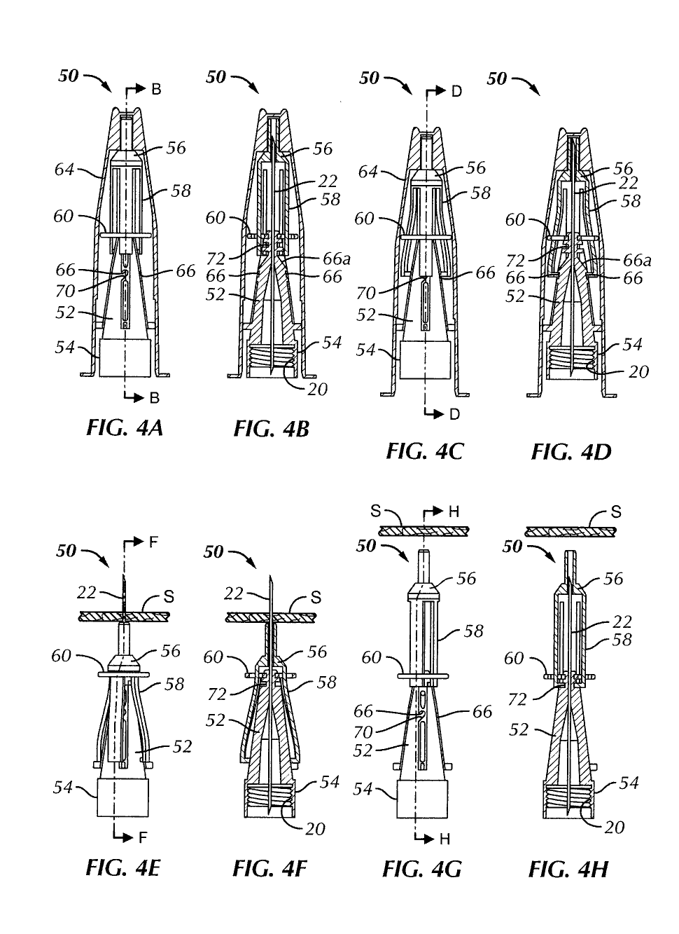

Referring to FIGS. 3-4H, the needle shield assembly 50 includes a needle hub 52 that has a conical or tapered outer surface 52a. A hollow cannula or needle 22 has first tip 22a for injecting into a patient and a second tip 22b for extending into the elastomeric seal on the distal end 26 of the vial cartridge 14 so that communication is established between the needle 22 and the vial cartridge 14. The needle 22 is attached to the needle hub 52 between the first and second tips 22a, 22b. A slideable sleeve 56 is engaged with the needle hub 52 by cantilever arms 58. The slideable sleeve 56 preferably includes four circumferentially equally spaced cantilever arms 58 that are each comprised of a resilient polymeric material but the slideable sleeve 56 may include any number of cantilever arms made of any other suitable material such as metal. The needle hub 52 and the slideable sleeve 56 each have receiving ends 52b, 56b which are distal to the first tip 22a and proximal the second tip 22a of the needle 22 and injection ends 52c, 56c which are proximal to the first tip 22a and distal the second tip 22a of the needle 22.

The needle shield assembly 50 includes a circular retaining ring 60, disposed around the cantilever arms 58 of the slideable sleeve 56. The retaining ring 60 is rotatably engaged with the distal end of the needle hub 52 and functions to retain the slideable sleeve 56 on the needle hub 52. The retaining ring 60 includes four evenly spaced apart holes or notches (not shown) for accommodating each of the four cantilever arms 58 of the slideable sleeve 56 with sufficient clearance to allow sliding and flexing of the cantilever arms 58 when captured within the retaining ring 60. The flexing of the cantilever arms 58 in combination with the retaining ring 60 creates a leaf spring-like arrangement of the slideable sleeve 56 when the slideable sleeve 56 is urged up and over the outer surface 52 a of the needle hub 52.

A packing sleeve or cover 64 is initially removably mounted on the needle hub 52 over the slideable sleeve 56. The cover 64 preferably includes a removable membrane 72 that encloses the cover 64 and seals the needle shield assembly 50 within the cover 64 during shipping, storage and initial handling of the needle shield assembly 50. The releasable membrane 72 is removable by grasping a tab (not shown) and peeling the releasable membrane off of the cover 64. The needle shield assembly 50 is preferably in an initial extended position (FIGS. 3, 4A and 4B) during shipping and leading up to the use of the needle shield assembly 50. In the initial extended position, there is little or no load on the cantilever arms 58 and the cantilever arms rest within an inwardly extending first path 66a in the needle hub 52. The injection end 56c of the slideable sleeve 56 preferably abuts a ledge, preferably in the form of a plurality of ribs 64a within the end of the packing sleeve 64. The ribs 64 form a receiving space 64b. An extension 56a preferably extends from the injecting end 56c of the slideable sleeve 56 and into the receiving space 64b. The extension 56a at least partially covers or shrouds the first tip 22a in the initial extended position. The first tip 22a of the needle 22 is preferably shrouded in the initial extended position (FIG. 3) but the first tip 22a may be partially or fully exposed when the needle shield assembly 50 is in the initial extended position. The top of the extension 56a is preferably open to allow the needle 22 to extend therethrough in subsequent positions.

The needle shield assembly 50 has a set or intermediate position (FIGS. 4C and 4D) where the cantilever arms are displaced outwardly preferably moments before using the needle shield assembly 50 to prevent creep of the cantilever arms 58 during shipping and storage. Alternatively, an elastic band or similar device (not shown) less susceptible to creep than the cantilever arms 58 may be wrapped around the cantilever arms 58 such that the intermediate position is not needed. As best seen by comparing FIGS. 4B and 4c, the first tip 22a of the needle 22 is preferably covered, but visible, by the extension 56a in the intermediate position either by making the extension 56a transparent, or by seeing the first tip 22a through the open end of the extension 56a. However, the first tip 22a may be partially or completely exposed in the intermediate position if it is preferred to see the bevel of the first tip 22a for example during injection.

To set the needle shield assembly 50 in the intermediate position, the needle hub 52 may be urged further into the cover 64 forcing the slideable sleeve 56 into contact with the plurality of ribs 64a, if they are not already in contact. Once the slideable sleeve 56 is in contact with the plurality of ribs 64a or the distal end of the cover 64, the slideable sleeve 56 remains stationary with respect to the cover 64 as the needle hub 52 continues to move toward the receiving space 64b. As the needle hub 52 moves relative to the slideable sleeve 56, the cantilever arms 58 slide up the needle hub 52 and are displaced outwardly by the tapered outer surface 52a of the needle hub 52 to thereby generate a restoring force that urges the slideable sleeve 56 away from the receiving end 52b of the needle hub 52. The cantilever arms 58 slide up the needle hub 52 (toward the receiving end 52b) until at least one of the cantilever arms 58 snaps over a catch 66. The catch 66 is an angled groove in the needle hub 52 that prevents retraction of the slideable sleeve 56 along the first path 66a in which the slideable sleeve 56 entered the catch 66. The needle hub 52 is prevented form further displacement relative to the slideable sleeve 56 by a shoulder 68 in the cover 64. The cover 64 may include one or more tracks (not shown) that engage with the needle hub 52 and prevent the cover 64 from being removed from the needle shield assembly 50 without first urging the needle hub 52 further into the cover 64 to set the needle shield assembly 50 in the intermediate position (FIGS. 4C and 4D). Though it is preferred that the cover 64 be used to set the needle shield assembly 50 into the intermediate position, the needle shield assembly 50 may be preset in the intermediate position or the needle shield assembly 50 may be set in the intermediate position without the use of the cover 64 such as with a user's hand.

Referring to FIGS. 1-2, when the pen injector 10 is assembled, the cartridge vial 14 at least partially containing liquid 14a is inserted into the vial holder 12 such that the amount of liquid 14a is visible through the window 12a. The vial holder 12 is then mounted to the dose setting apparatus 24. The vial holder 12 is preferably attached to the dose setting apparatus 24 by corresponding engaging threads 20b, 20c but the vial holder 12 may be mounted to the dose setting apparatus 24 in any manner such as snap fit. Alternatively, the vial holder 12 and the does setting apparatus 24 may be integrally formed such that the cartridge vial be inserted into the vial holder 12 through the distal end 18 of the vial holder 12 or through a hinged opening or door (not shown).

Referring to FIGS. 1-3, once the pen injector 10 is assembled, a new needle shield assembly 50 packaged in a cover 64 is selected and the membrane 72 is removed and discarded. The needle shield assembly 50 is then mounted onto the pen injector 10 by corresponding engaging threads 20a, 20b and the second needle tip 22b pierces the elastomeric seal of the vial cartridge 14. The needle 22 may also be positioned to only pierce the vial cartridge 14 of medication when the needle 22 is in use to prevent accidental discharge of liquid from the vial cartridge 14. The dosage is set, at any time in the process, by turning the knob 32 on the pen injector 10. The user then urges the needle hub 52 further into the cover 64 to set the needle shield assembly 50 into the intermediate position as shown in FIGS. 4C and 4D. Once the needle shield assembly 50 is set in the intermediate position the cover 64 is removed from the needle shield assembly 50. The extension 56a is pressed against the patient/user's skin S until the first tip 22a of the needle 22 is inserted into the skin S of the user. When a shot is to be administered, the needle shield assembly 50 is initially either in the initial extended position shown in FIGS. 3, 4A and 4B or the intermediate position shown in FIGS. 4C and 4D. Further movement of the needle 22 into the skin S urges the slideable sleeve 56 up the needle hub 52 (toward the receiving end 52b). Urging the needle hub 52 further into the slideable sleeve 56 causes the cantilever arms 58 to press against a cammed or slanted surface 70 extending from the grooves 66 to thereby twist or turn the slideable sleeve 56 relative to the needle hub 52 until the cantilever arms 58 align and engage with a second path 66b. The slanted surface 70 and the second path 66b form a generally helical track in which the cantilever arms 58 are forced to follow. A the needle 22 is pushed into the skin S, the slideable sleeve 56 continues to bear resiliently on the outer surface 52a of the needle hub 52 as the slideable sleeve 56 moves toward a retracted position where the first tip 22a extends more fully from the extension 56a and into the skin S. The slideable sleeve 56 may continue to slide up the outer surface 52a of the needle hub 52 in this manner to a fully retracted position (FIGS. 4E and 4F). Once the first tip 22a of the needle 22 is fully inserted in the skin S, the knob 32 is pressed to urge the piston 30 of the vial cartridge 14 toward the distal end of the pen injector 10 for a distance corresponding to the selected dose. Each time the knob 32 is depressed, the same dosage amount that was set is delivered through the needle 22.

Once the cantilever arms 58 of the needle shield assembly 50 enter the second path 66b and the injection end 56c of the slideable sleeve is released by withdrawing the needle 22 from the skin S, the slideable sleeve 56 moves or slides rapidly down the outer surface 52a of the needle hub 52 due to the stored resilient force. For example, when the needle 22 is withdrawn from the skin S, the resulting spring leaf force of the cantilever arms 58 in combination with the retaining ring 60 causes the slideable sleeve 56 to rapidly extend into a protective or the final extended position (FIGS. 4G and 4H) covering the tip of the needle 22 and thereby inhibiting accidental sticks with the needle 22 thereafter. During retraction of the needle 22, the slideable sleeve 56 bypasses the catch 66 because the cantilever arms 58 are now radially offset from the catch 66 (due to the prior rotation of the slideable sleeve 56) and are unimpeded as they slide down the needle hub 52. In the final extended position (FIGS. 4G and 4H), the cantilever arms 58 of the slideable sleeve 56 preferable engage with in a circumferentially extending annular lock groove 72 in the surface of the needle hub 52 to prevent the slideable sleeve 56 from exposing the needle 22 again. The needle shield assembly 50 is then removed from the pen injector 10 and is discarded. Once a second dose is needed, a new needle shield assembly 50 is installed. The amount of liquid 14a remaining in the vial cartridge 14 may be viewed by the viewing window 12a and marked with the dose markings 12b. The vial cartridge 14 may be replaced once the liquid 14a has been exhausted. The pen injector 10 may be reused while the needle shield assembly 50 and cover 64 are safely disposed of after each use.

It will be appreciated by those skilled in the art that changes could be made to the embodiment described above without departing from the broad inventive concept thereof. It is understood, therefore, that this invention is not limited to the particular embodiment disclosed, but it is intended to cover modifications within the spirit and scope of the present invention as defined by the appended claims.

I/We claim:

1. A pen injector comprising:
a vial cartridge;
a generally tubular body surrounding the vial cartridge, the body having a mounting end and a driving end;
a needle shield assembly removably mounted to the mounting end of the body, the needle shield assembly comprising:
a hollow needle having a tip for injecting into a patient, a needle hub at least partially surrounding the hollow needle, and a slideable sleeve slideably mounted on the needle hub, the needle hub and the slideable sleeve each having a receiving end which is distal to the tip of the needle and an injection end which is proximal to the tip of the needle,
wherein the receiving end of the needle hub is connected to the mounting end of the body and the slideable sleeve is slideable relative to the hub in the direction of the length of the needle between an extended position in which the tip of the needle is located completely inside the slideable sleeve and a retracted position in which the tip of the needle projects outwardly from the slideable sleeve,
the needle hub having an outer surface which deflects the slideable sleeve as the slideable sleeve slides toward the receiving end of the needle hub and into the retracted position, the slideable sleeve bearing resiliently on the outer surface of the needle hub during sliding movement of the slideable sleeve relative to the needle hub toward the retracted position such that a restoring force is generated for urging the slideable sleeve to move toward the injection end of the needle hub and into the extended position.

2. The pen injector of claim 1 further comprising a cover initially removably surrounding the hollow needle, needle hub and slideable sleeve of the needle shield assembly.

3. The pen injector of claim 2, wherein an injection end of the cover has an extended portion for receiving the needle tip when the slideable sleeve is in an intermediate position, the cover including at least one surface which abuts against the injection end of the slideable sleeve thereby causing the slideable sleeve to move to the intermediate position when the cover is urged toward the receiving end of the needle hub.

4. The pen injector of claim 2, wherein the cover is releasably mounted on the needle hub and covers the slideable sleeve such that the injection end of the cover covers at least the tip of the needle and the receiving end of the cover has an open portion to expose the receiving end of the needle hub, and by moving the cover in a direction toward the receiving end of the needle hub, the cover engages with the slideable sleeve causing the slideable sleeve to be retracted from the extended position to an intermediate position with respect to the needle hub.

5. The pen injector of claim 1, wherein the hub includes a groove that engages and releasably retains the slideable sleeve in an intermediate position between the extended position and the retracted position.

6. The pen injector of claim 5, wherein the needle tip is at least partially shrouded when the slideable sleeve is in the intermediate position.

7. The pen injector of claim 6, wherein the needle tip is completely shrouded in the intermediate position and is only exposed when the slideable sleeve is in the retracted position.

8. The pen injector of claim 1, wherein the vial cartridge is removable from the body.

9. The pen injector of claim 1, wherein the needle shield assembly further comprises a locking mechanism for retaining the slideable sleeve in the extended position after removal of the needle from the patient.

10. The pen injector of claim 1, wherein at least part of the receiving end of the needle hub has a tapered outer surface which tapers toward the injection end such that, in use, as the needle is inserted into a patient and the slideable sleeve slides toward the retracted position, the slideable sleeve is displaced outwardly by the tapered outer surface of the needle hub as the slideable sleeve slides toward the retracted position thereby generating the restoring force.

11. The pen injector of claim 1, wherein the outer surface of the needle hub is tapered and the slideable sleeve has at least one cantilever arm which bears resiliently on the tapered outer surface of the needle hub and, in use, as the needle is inserted into a patient and the slideable sleeve slides toward the retracted position, the at least one cantilever arm is displaced outwardly by the tapered outer surface of the needle hub thereby generating the restoring force.

12. The pen injector of claim 1, wherein the outer surface of the needle hub is tapered and the slideable sleeve has at least one cantilever arm which engages a helical track in the tapered outer surface of the needle hub such that, in use, as the needle is inserted into a patient and the slideable sleeve slides toward the retraced position, the at least one cantilever arm is displaced radially outwardly by the helical track thereby generating the restoring force.

13. The pen injector of claim 11, wherein the slideable sleeve has 4 cantilever arms.

14. The pen injector of claim 1, wherein the slideable sleeve has an initial extended position where the slideable sleeve is moveable toward the receiving end of the needle hub and a final extended position where the slideable sleeve is in a locked position.

15. The pen injector of claim 1, wherein the slideable sleeve, when in an initial position prior to use, is not under a substantial load.

16. The pen injector of claim 1, wherein the needle shield assembly is threadably attached to the body.

17. A pen injector comprising:
a vial cartridge having a piston;
a generally tubular body surrounding the vial cartridge, the body having a mounting end and a driving end, the driving end including a knob for setting a dosage level and driving the piston in accordance with the set dosage level;
a needle shield assembly removably mounted to the mounting end of the body, the needle shield assembly comprising:
a hollow needle having a first tip for injecting into a patient and a second tip for piercing the vial cartridge, a needle hub at least partially surrounding the hollow needle, and a slideable sleeve having a plurality of cantilever arms slideably mounted on the needle hub, the needle hub and the slideable sleeve each having a receiving end which is distal to the first tip of the needle and an injection end which is proximal to the second tip of the needle,
wherein the receiving end of the needle hub is connected to the mounting end of the body and the cantilever arms are slideable relative to the hub in the direction of the length of the needle between an extended position in which the first tip of the needle is located completely inside the slideable sleeve and a retracted position in which the first tip of the needle projects outwardly from the slideable sleeve,
the needle hub having a tapered outer surface which deflects the cantilever arms as the slideable sleeve slides toward the receiving end of the needle hub and into the retracted position, the cantilever arms bearing resiliently on the outer surface of the needle hub during sliding movement of the slideable sleeve relative to the needle hub toward the retracted position such that a restoring force is generated for urging the slideable sleeve to move toward the injection end of the needle hub and into the extended position.

18. The pen injector of claim 17, wherein the needle shield assembly includes a retaining ring disposed around the cantilever arms between the first tip of the needle and the injection end of the needle hub increasing the force that the cantilever arms exert against the outer surface of the needle hub.
